(19) United States
(12) Patent Application Publication
(10) Pub. No.: US 2006/0041244 A1
(43) Pub. Date: Feb. 23, 2006

(54) Hemocirculatory catheter and method of use thereof

(76) Inventors: Reinhard Hohmann, Hamburg (DE); Tommy Madsen, Hamburg (DE); Christian Schlensak, Gundelfingen (DE); Koppany Sarai, Ihringen (DE); Torsten Doenst, Freiburg (DE)

Correspondence Address: ALIX YALE & RISTAS LLP, 750 MAIN ST, HARTFORD, CT, 06103-2721, US

(21) Appl. No.: 11/103,804

(22) Filed: Apr. 8, 2005

(30) Foreign Application Priority Data
Apr. 8, 2004 (EP) 04 008 545.8

(51) Int. Cl. A61M 31/00

(52) U.S. Cl. 604/509; 604/104

(57) ABSTRACT

The catheter to be inserted into a vein (4) of the blood circulation, comprising a conduit (7) with at least one expandable anchoring element (13) at its distal end (8) for anchoring in the vein wall, and a tube-shaped jacket (sheet 14) that is pushed onto the conduit (7) and can be at least partially withdrawn in a proximal direction once the conduit (7) has been inserted into the vein (4), thereby releasing the at least one anchoring element (13), so that it can expand. The conduit (7) comprises a bottleneck (17) at the distal end of its lumen (8) and the catheter comprises a tube-shaped element (pusher 15) to be pushed onto a guiding wire (11). The front end of said element bearing against the bottleneck.

### FIELD OF THE INVENTION

The present invention generally relates to a device and a method of use which includes insertion of the device into anatomical structures associated with blood circulation. In particular, the invention relates to myocardial revascularization by partial arterialization of structures associated with the coronary sinus.

### BACKGROUND OF THE INVENTION

A consequence of coronary heart disease and the related narrowing or occlusion of coronary vessels is that coronary heart disease is a leading cause of death in industrialized countries. In fact, coronary heart disease is responsible for approximately 30% of deaths in these countries. In Germany, more than 340,000 persons die each year as a result of coronary heart disease. Aortocoronary bypass surgery is a conventional procedure for treating coronary heart disease, in particular if several coronary arteries are affected by the disease. Approximately 70,000 bypass operations are performed in Germany annually. About 60 per cent of the patients undergoing first-time bypass surgery are between 50 and 69 years of age.

As a general rule, aortocoronary bypass surgery is performed after opening the patient's thorax and connecting the patient to a heart-lung machine. Typically, the patient's heart is stopped during surgery for approximately 30 to 50 minutes once the principal artery has been clamped.

In spite of successful primary surgical treatment, coronary heart disease, in most cases, cannot be stopped and can spread even to the bypass graft. Typically, 30% of all bypasses are clogged again within 15 years after they were put in. This results in a second bypass being required in order to correct the clogged first bypass. This second bypass procedure includes a significantly increased surgical risk and a mortality rate of approximately 10%. The relatively high surgical risk associated with a secondary bypass surgical procedure is due to the following two factors: 1) possible lesion of the heart muscle or the larger vessels when the heart that is embedded in scar tissue is exposed, and 2) possible lesion of a still functional bypass during exposure of the heart, resulting in heart failure.

In addition, in the event of advanced coronary heart disease, it is possible that the path of the coronary vessels becomes altered to such an extent that it is impossible to sew a bypass to the coronary artery. Conventionally, there is not any established treatment for this group of patients. One attempted treatment is heart laser treatment, for example transynyocardial revescularization, for patients who could no longer be helped with conventional measures (bypass surgery), and is carried out only in exceptional cases. At the present time, there has been no evidence of any possible benefit for the patient.

In a study carried out at the Cleveland Clinic in 1999, the patient records of 500 angina pectoris patients who had undergone left heart catheter surgery during the three months of the study period were evaluated. Out of 500 patients, 59 (12 per cent) were not eligible for conventional treatment, i.e. bypass surgery (ACVB) or balloon dilatation (PTCA). Out of the 59, 21 received laser treatment. It is speculated that if the inclusion criteria for this study were expanded (patients with EF<25 per cent), the number of patients not eligible for treatment with traditional procedures would be greater.

Arterialization of heart veins was first described in 1948 by Beck as a possible treatment for patients who could no longer be treated with conventional surgery at the time. The principle was again taken up by Moll, who had already sutured a venous bypass on the coronary sinus. Surgery was performed using a heart lung machine. Consequently, the risks inherent in aortocoronary bypass surgery were not eliminated. Although Moll's initial description appeared to be promising, the technique was not widely accepted and was abandoned. The main argument against the technique described by Beck et al. was the development of a pronounced myocardial edema caused in Beck's technique by the total occlusion of the coronary sinus.

Presently, conventional application of this procedure involves advancing a tube into the coronary sinus and an end of the tube is anchored there. The free end of the tube is then drawn back through the right atrium. Following this, the free led out end of the conduit is connected with a central arterial blood supply. Thereby a blood flow is established from a central artery to the coronary sinus. This results in a blood flow whose direction is opposite that normally present in the coronary sinus.

However, for the conduit to fulfill its function, it is necessary to reliably anchor its distal end in a coronary vein. For this purpose one can use catheters of the type referred to in, for example, U.S. Pat. No. 6,406,491 B1. The catheter is inserted in the vein. Once it has reached its right position, the jacket is drawn back, thereby exposing the anchoring elements that anchor the distal end in the wall. The problem occurring in this case is that the anchor has to withstand the pulling force exerted when the jacket is drawn back. Occasionally, however, the sensitive vein is injured or the distal end of the conduit is torn from its anchoring place in the vein.

Catheters are known that comprise an actuator, a sheath and spreading elements, for example, U.S. Pat. No. 6,241,738 B1. However, these types of catheters are tools used for removing objects that have been inserted or are to be inserted in the vein. The spreading elements as taught and suggested by these catheters are not designed to engage the vein. In addition, those catheters are not conceived to remain in the body.

### SUMMARY OF THE INVENTION

Briefly stated, a catheter according to the present invention in a preferred form can be inserted in the coronary sinus or vein on a previously introduced guiding wire as would be done using catheters known in the art. When the jacket is withdrawn in order to expose the anchoring element(s), the pusher keeps the distal end of the conduit in place, such that pulling forces acting on the anchor are reduced. The pusher can then be pulled out of the conduit. In addition, strong pulling forces are reduced or eliminated since the pusher can slide on a guiding wire and inside the conduit without much resistance through use of, for example, suitable surface coating(s).

The catheter also includes a conduit with at least one expandable anchoring element at its distal end for anchoring in the vessel wall and a jacket that is associated with the conduit and which can be at least partially retracted in a proximal direction once the conduit has been inserted into the coronary sinus. The retraction thereby releases the at least one anchoring element, so that it can, for example, expand.

The anchoring elements can incorporate elastic elements, for example, elastic hooks that engage with the wall of the coronary sinus or vein once the jacket is withdrawn. The hooks may extend distally and outward from the conduit such that repositioning is possible by pushing the jacket and pressing the hooks together, following which the catheter may then be pushed in further or pulled out. The hook may alternatively extend proximally and outward. In compensation, one obtains greater safety against unintentional withdrawal, since the hooks, in this case, claw more firmly into the wall of the coronary sinus.

The hooks may have a rounded front end such that damage to the coronary sinus is reduced or avoided. In addition the hooks may be provided on only one side of the circumference of the conduit. This way they can be well anchored in, for example, the myocardium, whereas the other side of the coronary sinus, that is very thin, is not damaged.

The anchoring element may also be configured as a spiral that expands once the jacket has been withdrawn.

The pusher element may be made of plastic or carbon-like material, in particular polytetrafluorethylene; this has the advantage of especially low friction forces. Another advantageous embodiment is characterized in that the pusher is made of metal, in particular a very flexible, thin-walled metal tube, for example, similar to a canulla tube.

An engagement surface, for example a bottleneck, may be formed as part of the conduit and utilized as the engagement surface. The bottleneck may also be in the shape of a headpiece that is placed on the distal end of the conduit. In both cases, the bottleneck and the distal end of the pusher may be advantageously provided with complementary coupling devices. These coupling devices, by turning the pusher, allow the pusher to be coupled with the engagement surface, so that the pusher can apply not only a distally directed force, but also a proximally directed force for repositioning the catheter. The rotating coupling devices may be, for example, threads or bayonet couplings. The coupling part of the pusher with the conduit and/or a headpiece of said conduit can also be, for example, a unilateral eccentric shape.

The conduit may associate tightly at its distal end against the wall of the vein, so that no blood may flow in the opposite direction. In other cases, the conduit may be configured attached, and/or positioned to allow at least a certain backflow of blood in the opposite direction. For example, the distal end of the conduit may be positioned such that it does not tightly engage against the wall of the coronary sinus.

An object of the present invention in a preferred form is to create a catheter that will reduced the risk of disengaging the anchoring and thereby damage the vein when the jacket is withdrawn.

Another object of the invention is to provide a conduit with an engagement surface proximate its distal end, and to provide the catheter with a pusher that can be pushed on a guiding wire, a front portion of the pusher engaging with the engagement surface.

### BRIEF DESCRIPTION OF THE DRAWINGS

Other objects and advantages of the invention will be evident to one of ordinary skill in the art from the following detailed description with reference to the accompanying drawings in which:

FIGS. 1A and 1B are respectively schematic representations of the anterior and the posterior of a heart with its relevant blood vessels;

FIG. 2 is a schematic representation of an inserted conduit in relation to anatomical features consistent with the present invention;

FIG. 3 is a transverse cross-section of a distal end area of a catheter that is inserted in a coronary sinus before the jacket is withdrawn consistent with the present invention;

FIG. 4 is a transverse cross-section of a distal end area of a catheter as shown in FIG. 3 after the jacket has been partially withdrawn for releasing the anchoring elements consistent with the present invention;

FIG. 5 is a transverse cross-section of a distal end area of a catheter, showing an alternative embodiment of the anchoring elements consistent with the present invention;

FIG. 6 is a cut away side view of a headpiece with anchoring elements consistent with the present invention;

FIG. 7 is a transverse cross-section of a distal end area of a catheter having an alternative anchoring element consistent with the present invention;

FIG. 8 is a cut away side view of a conduit with a narrowed distal end consistent with the present invention;

FIG. 9 is a cut away side view of a rotating coupling device, a pusher and a headpiece consistent with the present invention;

FIG. 10 is a transverse cross-section of the distal end of a conduit consistent with the present invention; and

FIGS. 11-12 show a transverse cross-section and side view respectively of an alternative of the bottleneck and/or the rotating coupling device consistent with the present invention.

### DETAILED DESCRIPTION OF THE PREFERRED EMBODIMENT

For patients who can no longer be treated with conventional surgery techniques or for whom the risk involved in bypass surgery is extremely high, the invention in one embodiment includes a procedure of continuous retrograde coronary perfusion. In continuous retrograde coronary perfusion, excluding the use of a heart-lung machine, a small incision is made on the right side of the neck through which a catheter system is inserted in the cervical vein and advanced into the right atrium of the heart. The catheter system carries a conduit (plastic or autologous material) that is anchored in the coronary sinus by means of a special anchoring system. Following secure anchoring, the free end of the conduit is drawn back through the right atrium. Following this, the free led out end of the conduit is connected with the cervical artery that lies directly next to the cervical vein. Thereby blood flow is established from a central artery to the coronary sinus.

The present inventive surgical technique in one embodiment of the present invention provides partial arterialization of the coronary sinus. The conduit may be attached, for example, in or past the center third of the coronary sinus. This way, veinous inlets, for example, the V. interventricularis posterior (middle cardiac vein), the major vein draining blood from the right coronary vein and discharging only a few mm behind the ostium into the coronary sinus, remains open and continues to drain into the coronary sinus.

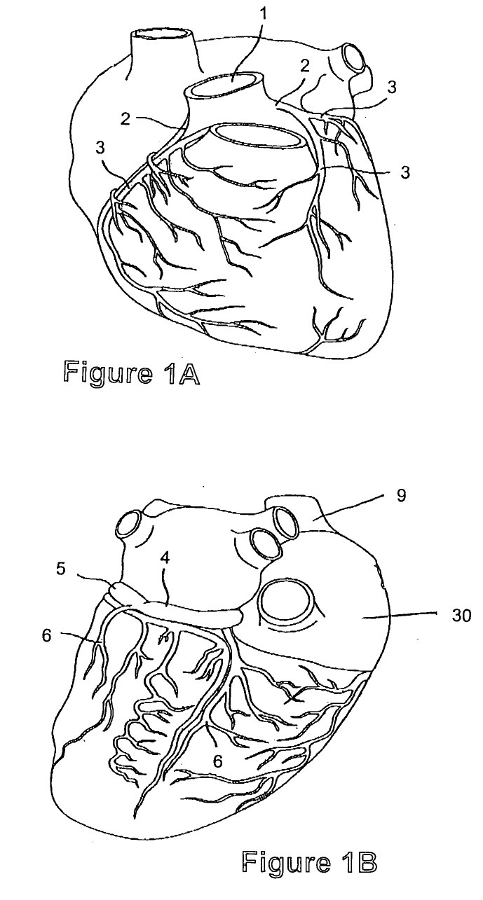

FIGS. 1A and 1B depict schematically a typical heart. From the aorta 1, blood flows, among other routes, through an opening into the coronary vessels 3, thereby supplying the heart muscles with among other things oxygenated blood. The blood flowing back from the heart muscle travels through the coronary vein 5 and various lateral branches 6, into the coronary sinus 4, and then into the right atrium 30. In cases of advanced coronary heart disease or surgery for recurrence, the paths of the coronary vessels may be altered to such an extent that it is technically no longer possible to sew a bypass on the coronary vein 5. For such patients it is possible to achieve revascularization of the coronary vessels by supplying the coronary vessels with arterial blood “from the rear”, i.e. via the coronary sinus. 4

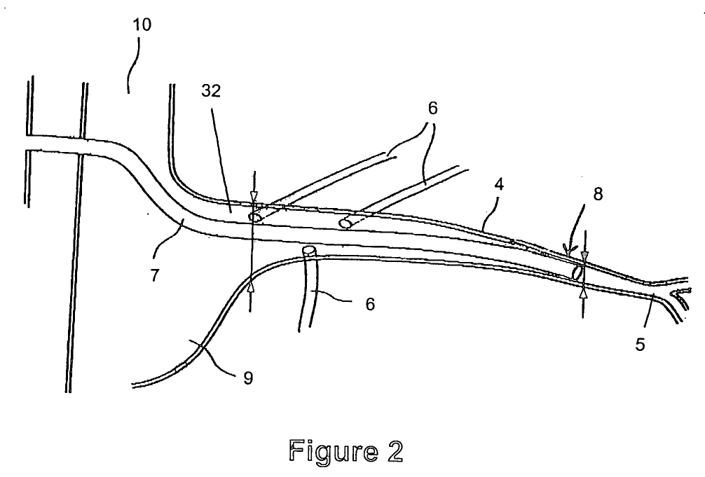

This revascularization can be achieved, in one embodiment of the present invention, as shown in FIG. 2, by a conduit 7 inserted through the ostium 32 of the coronary sinus 4. The conduit 7 extends from the coronary sinus into a large central and peripheral artery 10. For example, the cervical artery can be utilized. However, it should be understood that other arterial structures could be utilized. The conduit 7 passes through, for example, the right atrium and the superior vena cava 9. The transition from venous to arterial vessel is a well known surgical practice and as such does not have to be dealt with in detail in the present disclosure. However, the conduit 7 must be anchored by the distal end 8 of the conduit 7 so as to secure it in position.

It should be noted that this anchoring of the conduit 7 may be a tight association, since other veinous inlets, for example, small veins 6 lead into the coronary sinus 4, or may be of a less tight association, so as to maintain a certain backflow of venous blood.

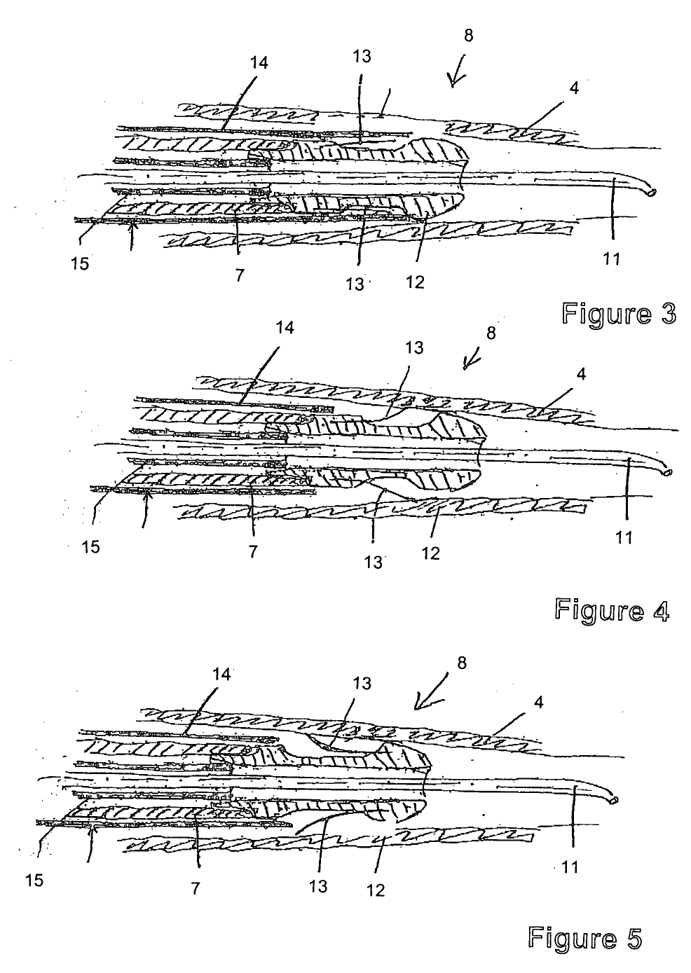

The invention includes several types of distal end 8 attachments. For example, as shown in FIG. 3, the distal end 8 of a conduit 7 is inserted in the coronary sinus 4. For this insertion, a guide wire 11 had previously been inserted in the coronary sinus 4. The conduit 7 is provided with a headpiece 12 at its distal end 8. Anchoring elements 13, for example, hook shaped anchoring elements, are provided on the outer circumference of the headpiece 12. A pusher 15 bears on said headpiece 12. The headpiece 12 may extend as far as the proximal end of the catheter. The pusher 15, for example, exerts a counter force on the headpiece 12 when a jacket 14 associated with the anchoring elements 13 is withdrawn in order to release the anchoring elements 13. This, among other things, prevents the distal end 8 of the conduit 7 from unintended withdrawal at the same time the jacket 14 is withdrawn. Unintended withdrawal of the anchoring elements 13 could, among other things, lead to the wall of the coronary sinus 4 being damaged and/or the distal end 8 being removed from its desired location. It should be noted that the conduit 7 may be anchored proximate to or in the vein 5 in embodiments of the present invention.

In one embodiment of the present invention, as is shown in FIG. 4, the anchoring elements 13 have elastic properties such that they may spread apart through utilization of the elastic properties. Upon spreading out, the anchoring elements 13 associate with the wall of the coronary sinus 4. In operation, for example, once the jacket 14 is proximally retracted far enough that the conduit is exposed, the pusher 15 may also be pulled out. By choosing suitable materials and/or surface treatments of the various elements, friction against the guide wire 11 and the conduit 7 can be sufficiently reduced. This can enhance the prevention of a damaging pull force being exerted on the anchoring elements 13.

In one embodiment of the present invention, as shown in FIGS. 3 and 4, the anchoring elements 13 spread apart in a substantially distal direction. This allows for the jacket 14 to be brought into association with the anchoring elements such that the anchoring elements 13 are compressed toward the headpiece 12. This allows for the distal end 8 of the conduit 7 to be, for example, repositioned.

In one embodiment of the present invention, as shown in FIG. 5, the anchoring elements 13 spread laterally in a substantially proximal direction. This proximal spreading, for example, provides a better hold which provides, among other things, greater resistance to withdrawal. The disadvantage of this backward spreading is, for example, that the anchoring elements 13 cannot generally be pressed together again by pushing the jacket 14 over them.

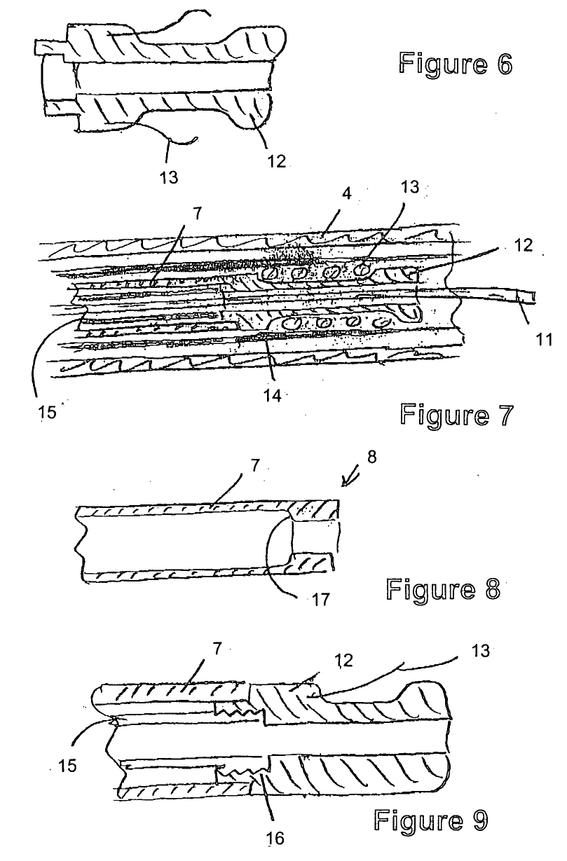

In one embodiment of the present invention, as shown in FIG. 6, the ends 100 of the anchoring elements 13 are rounded so that, for example, the risk of damaging the wall of the coronary sinus 4 is lessened.

In one of the embodiment present invention, as shown in FIG. 7, an anchoring element 13 positioned in the headpiece 12 is provided in the form of a spiral. In operation, wherein the jacket 14 is retracted in a proximal direction, the spiral formed anchoring element 13a expands laterally outward from the headpiece 12 such that coils of the spiral formed anchoring element contact the adjacent coronary sinus 4. Among other things, the spiral formed anchoring element 12a distributes contact pressure along a length of the coronary sinus 4 via the coil contact points. This can reduce or eliminate damage to the coronary sinus 4.

In one embodiment of the present invention, as shown in FIG. 8, the distal end 8 of the conduit 7 includes a narrowed portion 17 against which the pusher 15 may come to bear. The narrowed portion 17 allows for, among other things, a positive control surface upon which the pusher 15 can engage and be disengaged from.

In one embodiment of the present invention, as shown in FIG. 9, the device includes a rotating coupling device 16 which couples the pusher 15 to the headpiece 12. The coupling device allows for an engagement of the pusher 15 and the headpiece 12 such that the pusher 15 can also exert a proximally directed force on the headpiece 12. This rotating coupling 16 can include, for example, a thread or a bayonet coupling (shown in FIGS. 11 and 12). The pusher 15 and headpiece 12 may be disengaged by a selective rotation of the pusher 15 relative to the headpiece 12.

In one embodiment of the present invention, as shown in FIG. 9, the anchoring elements 13 may be arranged on selected portions of the headpiece 12, for example, on substantially only one side of the headpiece 12. This arrangement allows for the anchoring elements 13 to, for example, be anchored into the myocardium with the other side being proximate to delicate structures, such as thin coronary sinus or vein walls. This aids in reducing damage to the delicate structures which might otherwise occur if they were contacted by the anchoring elements 13.

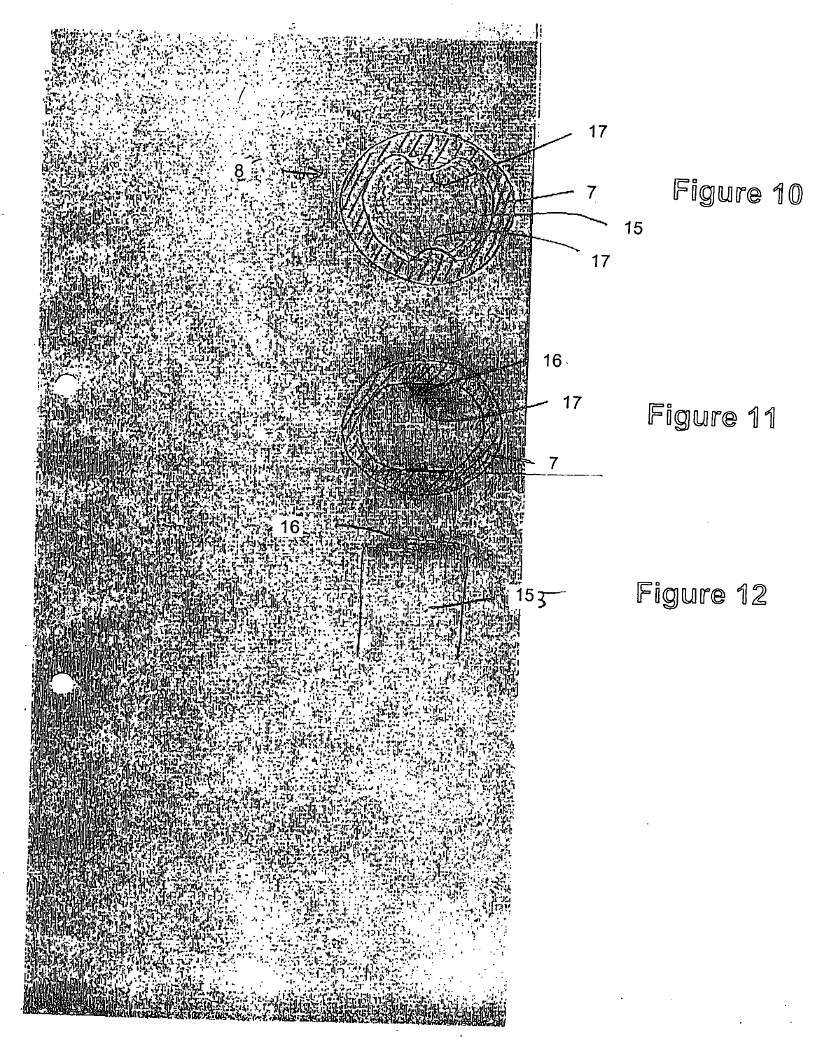

In one embodiment of the present invention, the distal end 8 of the conduit 7 is shown in FIG. 10. The narrowed portion 17a does not extend around the entire inner circumference of the conduit, but substantially consists of two stops against which the pusher 15 comes to bear. This way, the portion 17a restricts the lumen 34 of the conduit 7 to a lesser degree which results in a lessened resistance to the blood flow entering the conduit.

In one embodiment of the present invention, as shown in FIG. 11, the narrowed portion 17b consists substantially of peg-like stops 16 of a bayonet coupling whose complementary recesses at the end of the pusher 15 are shown in FIG. 12.

It should be understood that the following examples are included for purposes of illustration so that the invention may be more readily understood and are in no way intended to limit the scope of the invention unless otherwise specifically indicated.

Exemplary Experimental Procedures

Four experimental procedures were carried out in a manner consistent with the present invention. The experimental procedures were carried out using domestic pigs. Experimentally several pigs were obtained and anesthetized and monitored:

Five experiments were performed, three of which were successful (cf. below). In two animals the experiment had to be aborted. In both animals, coronary sinus perfusion was unable to sufficiently perfuse the myocardium following occlusion of the coronary arteries. Both animals died of acute heart pump failure shortly after extra-corporal circulation was stopped.

The detailed inspection of the coronary arteries and veins of the animals which died of heart pump failure showed that in both cases, the animals had an anomaly in their coronary veins. In either animal, only two coronary sinus ostia were identified in the atrium. The coronary veins of the R. interventricularis drained into one ostium, the vein of the A. circumflexa into the other. During the experiment, coronary perfusion occurred only through one ostium. This anomaly explains the failure of the experiments.

Subsequently 20 pig hearts were examined from butchered animals. All 20 pig hearts had a coronary sinus in atypical location into which drain all three major heart veins. One can assume that the anatomical variation of the coronary sinus discovered in the pilot test was an accidental result. Such variations have so far not been reported in humans.

The pilot tests showed that retrograde perfusion of the coronary veins alone, using arterialized blood is sufficient for adequate myocardial perfusion.

A phase 1 surgery was performed in order to create chronic ischemia. The Phase 1 surgery was performed by the surgical steps which included:

Placing a pig in lateral position; putting in an arterial pressure line in A. femoralis; putting in a venous shunt for flow-directed insertion of a pulmonary catheter; placing a Millar catheter; performing a lateral thoracotomy; exposing the RIVA immediately below bifurcation of a first diagonal branch; applying a circular stenosis of the coronary artery by means of a Dacron™ band wrapped around the vessel and sutured to itself so as to reduce the blood flow through the coronary artery by approximately 50% (controlled by flow measurement).

In addition, the following parameters were measured: RR interval (syst/mean/diast); ZVD; HF; Cl; HZV; PAP (syst/mean/diast); PCWP; PVR; SVR; and Blood gas analysis (lactate, pH).

Furthermore, laboratory chemical analyses were conducted which included testing LDH, HBDH, lactate, and Epicardial echocardiography were measured.

Next, injection of microspheres was carried out along with (Time 1) measuring with first color for initial reference value, 5 million (first color) in LA as bolus for 30 sec; reference sample from A. carotis: 5 sec. before start of bolus injection; aspiration for 90 sec., 7.5 mL/min.). Other tests included measuring (catecholamines, defibrillation, instability, etc.)

A Phase 2 surgery was subsequently performed in order to place a coronary sinus bypass. The Phase 2 surgery was performed by surgical steps which included:

Placing the pig in a supine position, putting arterial pressure line in A. femoralis; putting in a venous shunt for flow-directed insertion of a pulmonary catheter; placing a Millar catheter; performing a median sternotomy; creating a longitudinal opening of the pericardium; disposing a ligature on left side in permanent V. cava superior; administering systemic heparinization (ACT approximately 500 sec); connecting to a pig heart-lung machine (HLM) with canullation of A. ascendens and direct canullation of V. cava superior (SVC) and V. cava inferior (IVC); starting cardiopulmonary bypass (routine CPB priming with crystalloid solution); harnessing SVC and IVC; opening up right atrium in the beating heart; inserting a 5 mm PTFE tubular prosthesis about 3 to about 4 cm into the coronary sinus (a Palmas stent is attached to distal end of a prosthesis); attaching a prosthesis in coronary sinus following ballooning of stent with a balloon catheter; adjusting the length of tubular prosthesis, tangentially clamping A. descendens or Truncus brachiocephalicus; end-side anostomosing of the tubular prosthesis with A. ascendens or Truncus brachiocephalicus with a continuous suture; prosthesis initially remains clamped; suture closure of right atrium; opening coronary sinus bypass; and disconnecting HLM. The following parameters were measured: RR (syst/mean/diast); ZVD; HF; Cl; HZV; PAP (syst/mean/diast); PCWP; PVR; SVR; and Blood gas analysis (lactate, pH).

In addition, chemical analyses were carried out in the laboratory, for example, LDH, HBDH, lactate. Also measured was the flow in the coronary sinus bypass, Epicardial echocardiography results before and after placement of coronary sinus and bypass

There was an injection of microspheres, (Time 1) measuring with one color each time, 5 million in LA as bolus for 30 sec; reference sample from A. carotis: 5 sec. before start of bolus injection; aspiration for 90 sec., 7.5 mL/min.). In addition, the following time point actions were taken:

Time 2: myocardial perfusion only through native coronary system (coronary sinus open); Time 3: myocardial perfusion only through native coronary system after insertion of CS bypass in coronary sinus; Time 4: myocardial perfusion only through CS bypass after occulsion of native coronary arteries; and other (catecholamines, defibrillation, instability, etc.)

A Phase 3 surgery was subsequently carried out in order to access cadial function, analyze perfusion, and remove selected organs.

The phase 2 surgery was performed by surgical steps which included:

Placing the pig in a lateral position; putting in an arterial pressure line in the A. femoralis; putting in a venous shunt for flow-directed insertion of a pulmonary catheter; placing a Millar catheter; performing a median thoracotomy; creating a longitudinal opening in the pericardium; exposing the heart; measuring the following parameters: RR (syst/mean/diast), ZVD, HF, Cl, HZV, PAP (syst/mean/diast), PCWP, PVR, SVR, Blood gas analysis (lactate, pH).

In addition, chemical analyses in laboratory (LDH, HBDH, lactate); measuring flow in coronary sinus bypass; and Epicardial echocardiography.

An injection of microspheres was made, wherein at Time 1 a reference value was determined by measuring with one color, 5 million (fifth color) in LA as bolus for 30 sec; reference sample from A. carotis: 5 sec. before start of bolus injection; aspiration for 90 sec. 7.5 mL/min.)

The pig was then euthanized with a KCL infusion at end of test.

Removal of organs and sample storage/processing was then done as follows: Heart: analysis of microsperes; Lung; and Kidney.

In summer 2004, we carried out four pilot tests on pigs with partial arterialization of the coronary sinus were performed.

Additional Exemplary Experimental Procedures

Additional experimental procedures which included a longer follow-up period were performed on pigs. For this, the first step was to create chronic ischemia of the heart muscle by creating a severe stenosis of the R. interventricularis and R. circumflexus. The degree of stenosation of the coronary artery is measured by means of flow monitoring and angiography.

Subsequently, the animals were monitored for seven days. After seven days, the animals were operated on a second time. As described below, the coronary sinus bypass was then put in place by means of median sternotomy using the HLM. After weaning from HLM, function analyses was carried out on the still anesthesized animals (cf. below). Following this, the animals, after a monitoring period of four weeks, were again anesthesized for another series of measurements for cardiac function and perfusion. The animals were then euthanized for organ removal for pathological evaluation.

The additional exemplary experimentation was divided in three phases as follows:

A Phase 1 surgery wherein chronic myocardial ischemia was created. After surgery, the animals woke up and were monitored for seven days. The animals were given post-operative analgesics. In the further course of the test, the animals were monitored on a daily basis. In the event of severe cardiac complications or other life-threatening impairments, the animals were immediately euthanized by a veterinarian;

A Phase 2 surgery wherein after seven days, the animals were operated on once more with the coronary sinus bypass being put in place. While the animals were still under anesthesia, the necessary function analyses was performed. The animals were given post-operative analgesics. After this surgery, the animals were also checked up on daily; in the event of life-threatening impairments, the animals were euthanized by a veterinarian; and

A Phase 3 surgery wherein after a convalescence phase of four weeks, the third and last surgery was performed under anesthesia, also for performing function analyses. The animals were then euthanized. At the end of the examination, the animals did not awaken from the anesthesia; irreversible heart failure was induced by potassium injection.

It should be noted that the anatomy of the heart of the domestic pig is closest to that of humans. Findings from experiments on pig hearts can generally be transferred to clinical practice in humans.

The present animals test project was carried out on 20 domestic pigs of a size ranging from about 30 kg to about 40 kg.

Initially, the impact of the above-mentioned surgical procedure was to visually examine the open heart. Following a median sternotomy, the animals were hooked up to the HLM. Following an opening of the right atrium, a plastic conduit (5 mm tubular prosthesis) is inserted in the beating heart. The distal end of the conduit was attached in the coronary sinus with an anchoring element. The conduit was then led out of the atrium and anastomosed with the A. ascendens. The atrium is closed again. The permanent V. cava superior, located in pigs on the left, draining into the coronary sinus, is ligated. Once the blood flow is opened through the coronary sinus conduit, the left and right coronary veins are progressively closed by means of clips. Disconnection of HLM is made wherein there is a termination of extra-corporal circulation.

All surgical steps were carried out in sterile conditions. After a 12 hours without feeding, the animals were anesthesized with midolazam (Dormicum®) (intravenous infusion of 0.2 mg/kg/h), fentanyl (intravenous infusion of 5-10 μg/kg/h) and vecuronium (Norcuronh®) (intravenous infusion of 0.05-0.1 mg/kg/h) and endotracheally entubated. 10 mL/kg/h of Ringre's bicarbonate solution were infused as a basal volume substitution. The respiratory minute volume and respiratory frequency were set in such way that the arterial PO2 was between 90 mmHg and 110 mmHg and the arterial PO2 between 38 mmHg and 42 mmHg. During mechanical ventilation, a positive endexpiratory pressure (PEEP) of 5 mmHg was applied. When a CPB was put in, the lung was not ventilated, but PEEP of 5 mmHg was applied to prevent an alveolar collapse. During surgery, the animals were placed on a heating pad. Rectal temperature was maintained at between 36° C. and 37° C. During the entire surgery, arterial blood pressure (catheter in A. carotis), heart frequency, ECG, and blood gas parameter were continuously monitored. At the end of the experiments, the animals were euthanized by intravenous injection of potassium chloride (20 mmol/kg).

Phase 1 surgery wherein chronic ischemia was induced included the surgical steps of placing the pig in a lateral position; performing a lateral thoracotomy; exposing the R. interventricularis anterior immediately below the bifurcation of first diagonal branch; creating a circular stenosis of coronary artery by means of a Dacron™ band wrapped around the vessel and sutured to itself in order to reduce blood flow by approximately 50% (controlled by flow measurement).

It should be noted that in order to evaluate changes in contractility and hemodynamics prior to and after stenosation of the coronary artery, the following parameters were determined in each anesthesized animal in each case prior to and after surgery:
1) Laboratory analyses (LDH, HBDH, lactate)
2) Hemodynamics:
Measurement of contractility by means of impedance and Millar catheter
Pulmonary catheter for determining heart-time volume, cardiac index, peripheral and pulmonary vessel resistance
Epicardial echocardiography
3) Microspheres, Time 1: measuring with one color as initial reference value.

This data served as process parameters for cardiac function for subsequent surgery.

Phase 2 surgery wherein a coronary sinus bypass was created included the surgical steps of placing the pig in a supine position, median sternotomy; creating a longitudinal opening in pericardium; placing a ligature on left side in permanent of the V. cava superior; administering systemic heparinization (ACT approximately 500 sec); connecting pig to heart-lung machine (HLM) with canullation of A. ascendens and direct canullation of V. cava inferior (SVC) and V. cava inferior (IVC); starting cardiopulmonary bypass (routine CPB priming with crystalloid solution); harnessing SVC and IVC; opening up right atrium in beating heart; inserting a 5 mm PTFE tubular prosthesis 3 to 4 cm into coronary sinus (a Palmas stent is attached to distal end of prosthesis); attaching prosthesis in coronary sinus following ballooning of stent with balloon catheter; adjusting length of tubular prosthesis, tangentially clamping of A. descendens or Truncus brachiocephalicus; end-side anostomosing of tubular prosthesis with A. ascendens or Truncus brachiocephalicus by means of continuous suture; prosthesis initially remains clamped; suture closure of right atrium; opening coronary sinus bypass.

In addition, the animals were connected to the CPB for 30 min. During this period, the coronary sinus bypass was anastomosed. At the end of the CPB, the animals were monitored. Experimental parameters included Survival after CPB; Hemodynamic measurements (Measurement of contractility by means of impedance and Millar catheter; Pulmonary catheter for determining heart-time volume, cardiac index, peripheral and pulmonary vessel resistance; and Epicardial echocardiography); Laboratory analyses (LDH, HBDH, lactate); and Determination of regional myocardial perfusion by means of fluorescent microspheres prior to and after placement of coronary sinus bypass.

The measurement of myocardial perfusion using fluorescent microspheres was performed during the CPB according to the described technique. Measurement of blood flow using ms was based on the principle that the microspheres, after a single passage through the system cling to the terminal capillary vessel. When they are removed from the respective tissue, they can be counted. Based on the ration of circulating microspheres in the blood (blood reference value) and the number of microspheres in the tissue, one can determine regional blood flow. During CPB, the microspheres are injected at three moments:
1) Time 2 (second color): myocardial perfusion only through native coronary system (coronary veins are stenosed; coronary sinus is open)
2) Time 3 (third color): myocardial perfusion only through native coronary system following placement of coronary sinus bypass in the coronary sinus. Coronary sinus still occluded.
3) Time 4 (fourth color): myocardial perfusion through open coronary sinus bypass.

Each microsphere injection contains 2 million microspheres. Three different colors were used for each of the three measuring periods (“blue-green”, “orange”, “yellow”). The microspheres were dissolved in 4 mL of NaCl/TWEEN 80 (solution ratio: 1 drop of TWEEN 80 per 100 mL of NaCl) and injected into the left atrium as bolus over a period of 5 sec. Aspiration of blood sample from the A. descendens (blood reference sample) was started five minutes prior to injection of microspheres. Aspiration was done for 90 sec at a flow of 7.5 mL/min using a mechanical precision pump (Harvard pump 22, FMI GmbH, Ober Beerbach, Germany). The blood was aspirated through a 40 cm long Teflon-coated catheter and collected in a 20 mL glass syringe (Hero, Germany). Prior to this, the Teflon catheter was advanced to the level of the A. descendens through a shunt system lying in the abdominal aorta. The flask of the glass syringe had previously been coated with TWEEN 80 (polyoxyethylene sorbital mono-oleate, Sigma Chemical, St. Louis, USA). In addition, 2 mL of heparin (10,000 I.E.) were put in the syringe. This prevented both the aggregation of the microspheres within and at the needle, and coagulation of the aspirated blood. The syringe was weighed prior to and after aspiration in order to calculate the amount of aspirated blood with reference to blood density (1.055). The actual microsphere concentration per mL of blood was then calculated using the blood reference value.

For calculating myocardial blood flow for each sample, the following formula was used (MS=microspheres; HTV=heart-time volume; reference=blood reference sample):
Blood flow [mL/min]=MS myocardium×HTV[mL/min/MS reference

The sum total of blood flow readings from all myocardial samples was the total blood flow in the myocardium.

Phase 3 surgery wherein cardiac function, perfusion analysis, and organ removal was accomplished included the steps of placing the pig in supine position; performing a median sternotomy; creating a longitudinal opening in pericardium; exposing the heart; performing function and perfusion analysis.

At the end of the experiment, the entire heart was removed and divided into three parts: right ventricle, septum, left ventricle. The number of microspheres in each of the three muscle preparations was analyzed (Fa. Perfusion Technologies Ltd., Freiburg, Germany).

After removal, the heart muscle tissue was weighed, dried for 48 hours at 80° C. in a drying furnace, and then weighed again. The ratio of moist to dry matter was calculated and used as marker for water content in the heart muscle.

As a final measure, the following parameters were determined for evaluating cadiac contraction and perfusion:
1) Laboratory analyses (LDH, HBDH, lactate)
2) Hemodynamic measurements:
Measurement of contractility by means of impedance and Millar catheter
Pulmonary catheter for determining heart-time volume, cardiac index, peripheral and pulmonary vessel resistance
Epicardial echocardiography
3) Determination of regional myocardial perfusion using fluorescent microspheres.

It should be noted as observational validation of the technique that intraoperative findings in the coronary arteries and coronary veins of a pig following ligature of R. interventricularis and R. circumflexus and initiation of coronary sinus perfusion showed that the observed bright red vessels are the coronary veins through which arterialized blood was flowing, and that the observed dark red vessels were the coronary arteries through deoxygenated blood was flowing into the aortic root.

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and the scope of the present invention.

What is claimed:

1. A catheter to be inserted into a vein of blood circulation for myocardial revascularization by partial arterialization of a coronary sinus, comprising:
a conduit having a lumen and with at least one expandable anchoring element at its distal end for anchoring in a vessel wall, the conduit comprising a bottleneck at the distal end of its lumen;
a tube-shaped jacket that is pushed onto the conduit and can be at least partially retracted in a proximal direction once the conduit has been inserted into the vessel, thereby releasing the at least one anchoring element, so that it can expand; and
a pusher to be pushed onto a guiding wire, a front end of said pusher bearing against the bottleneck.

2. The catheter according to claim 1, characterized in that several anchoring elements are provided in the form of elastic elements.

3. The catheter according to claim 2, characterized in that said elastic elements are hooks which extend distally and outwardly from said conduit.

4. The catheter according to claim 2, characterized in that said elastic elements are hooks which extend proximally and outwardly from said conduit.

5. The catheter according to claim 2, characterized in that said elastic elements are hooks rounded at their front end.

6. The catheter according to claim 2, characterized in that said elastic elements are hooks arranged on only one side of the circumference of said conduit.

7. The catheter according to claim 1, characterized in that the anchoring element is a spiral.

8. The catheter according to claim 1, characterized in that the pusher is made of plastic or plastic-like material.

9. The catheter according to claim 1, characterized in that the pusher is made of polytetrafluorethylene.

10. The catheter according to claim 1, characterized in that the pusher is made of flexible, thin-walled metal.

11. The catheter according to claim 1, characterized in that the bottleneck in the lumen is formed in the distal end area of the conduit.

12. The catheter according to claim 1, characterized in that the conduit comprises a headpiece provided with said bottleneck at its distal end.

13. The catheter according to claim 1, characterized in that said bottleneck and the distal end of the pusher are provided with complementary rotating coupling devices.

14. The catheter according to claim 13, characterized in that said complementary rotating coupling devices are threads.

15. The catheter according to claim 13, characterized in that said complementary rotating coupling devices are bayonet couplings.

16. The catheter according to claim 1, characterized in that said bottleneck and/or the rotating coupling devices extend only over part of the inner circumference of said conduit.

17. A method of myocardial revascularization by arterialization comprising:
accessing a coronary sinus having a first end, a second end, and an intermediate portion having a veinous inlet, said second end fluidly connected to a vein having a lumen defined in part by a wall;
inserting a distal end of a conduit having a proximal end, a central lumen, and a distal end through the coronary sinus into a portion of the coronary sinus or into a portion of the vein;
engaging the distal end of the conduit with the coronary sinus or the vein;
connecting the proximal end of the conduit to a central arterial blood supply; and
supplying a substantially continuous blood flow from the central arterial blood supply through the central lumen of the conduit into the portion of the coronary sinus or the vein.

18. The method of myocardial revascularization by arterialization of claim 17, wherein the step of engaging the distal end of the conduit with the coronary sinus or vein:
a pusher;
engaging an engagement portion of the pusher with an engagement surface of the conduit;
advancing the conduit to a selected location in the portion of the coronary sinus or the vein;
stabilizing the conduit at the selected location with the pusher; and
moving a jacket in a direction relative to the conduit such that an anchoring element engages a portion of the portion of the coronary sinus or the vein.

19. The method of myocardial revascularization by arterialization of claim 17, wherein the coronary sinus is accessed with a hemocirculatory catheter comprising:
a conduit which further includes an engagement surface proximate the conduit distal end, and a headpiece having an elastic anchoring element which exerts a lateral bias force outwardly from the headpiece;
a pusher removably disposed within a central lumen of the conduit, said pusher having a proximal end, a distal end, and an engagement portion proximate the pusher distal end, said engagement portion being engageable with the conduit engagement surface;
a jacket having a distal end and being removably disposed about an exterior of the conduit such that when the jacket is in an insertion position the anchoring element is compressed toward the headpiece by the jacket.

20. The method of myocardial revascularization by arterialization of claim 18, further including the steps of:
moving the jacket in a direction relative to the conduit such that the anchoring element is disengaged from the portion of the coronary sinus or the vein; and
repositioning the conduit in the portion of the coronary sinus, or the vein.

21. The method of myocardial revascularization by arterialization of claim 19, wherein the headpiece defines a portion of the conduit engagement surface.

22. The method of myocardial revascularization by arterialization of claim 18, wherein the conduit engagement surface and the pusher engagement portion form a bayonet connection.

23. The method of myocardial revascularization by arterialization of claim 17, further including the steps of:
identifying a delicate portion of the portion of the coronary sinus or the vein; and
orienting a portion of the conduit distal end without an anchoring element proximate the delicate portion of the portion of the coronary sinus or the vein.

24. The method of myocardial revascularization by arterialization of claim 17, wherein the coronary sinus is accessed with a hemocirculatory catheter in a step which includes guiding the catheter along a guide wire.

25. The method of myocardial revascularization by arterialization of claim 19, which includes modifying the frictional coefficient of portions of the catheter such that a disengagement force exerted on an anchoring element is reduced.

26. The method of myocardial revascularization by arterialization of claim 17, wherein the step of supplying a substantially continuous blood flow from the central arterial blood supply through the central lumen of the conduit into the portion of the center third of the coronary sinus or the vein includes:
preventing ischemia in myocardial tissue.

27. The method of myocardial revascularization by arterialization of claim 17, wherein the step of engaging the distal end of the conduit with the portion of the coronary sinus, or the vein includes the step of providing a substantially incomplete seal between the conduit and the portion of the coronary sinus or the vein.

28. The method of myocardial revascularization by arterialization of claim 17, wherein the step of engaging the distal end of the conduit with the coronary sinus or the vein includes the step of providing a substantially complete seal between the conduit and the portion of the coronary sinus or the vein.

29. The method of myocardial revascularization by arterialization of claim 17, wherein the step of engaging the distal end of the conduit with the coronary sinus or the vein includes selectively expanding an anchoring element against a wall of the portion of the coronary sinus, or the vein.

30. The method of myocardial revascularization by arterialization of claim 17, further including the step of flowing veinous blood from the veinous inlet, past an exterior of the conduit, and out the first opening of the coronary sinus.
